(19) United States
(12) Patent Application Publication
(10) Pub. No.: US 2024/0394825 A1
(43) Pub. Date: Nov. 28, 2024

(54) EYE SURGERY VISUALIZATION SYSTEM, AND OPERATING METHOD AND COMPUTER PROGRAM

(71) Applicant: CARL ZEISS MEDITEC AG, Jena (DE)

(72) Inventors: Christoph HAUGER, Oberkochen (DE); Thomas LANG, München (DE); Frank DEUBEL, Oberkochen (DE)

(21) Appl. No.: 18/671,283

(22) Filed: May 22, 2024

(30) Foreign Application Priority Data
May 22, 2023 (DE) 102023113284.9

(51) Int. Cl. G06T 1/00; A61B 34/20; A61F 9/007; G06T 7/00

(52) U.S. Cl. CPC G06T 1/0007; A61F 9/007; G06T 7/00; A61B 2034/2057; G06T 2207/30041

(57) ABSTRACT

An eye surgery visualization system, an operating method, and a computer program with a program routine for registering a patient's eye model to a patient's eye based on a patient's eye registration structure and a model registration structure, in which object region image data related to an image of the patient's eye captured by an image capture device is linked to optical imaging parameters of the image capture device to provide in the form of data a patient's eye coordinate system which is fixed in relation to the patient's eye and referenced to a model coordinate system.

The invention relates to an eye surgery visualization system having a device for the stereoscopic visualization of an object region with an operating site on a patient's eye, the device for the stereoscopic visualization of an object region comprising an image capture device for capturing object region image data containing image data related to at least one patient's eye registration structure, having a computer unit, and having a biometry data interface for the pro-vision of patient's eye biometry data, which define a patient's eye model having a model registration structure corresponding to the patient's eye registration structure and a model coordinate system.

An operating system of the type set forth at the outset is known from DE 10 2020 102 011 A1. This eye surgery operating system contains an OCT apparatus which serves for capturing the position of a surgical tool in a model of a patient's eye, which can be displayed to a surgeon as a 3-D reconstruction of a region of the patient's eye. The display unit allows for the display of an actual and a target position for the surgical tool.

Displaying to a surgeon a piece of position information referenced to a patient's eye and related to a surgical incision or surgical incisions within the scope of an eye surgery operating system was disclosed in DE 10 2018 124 065 A1.

US 10 842 573 B2 describes an eye surgery operating system containing a computer unit for creating a calculation model for assisting eye surgeons, the model allowing an estimation of the load on the retina during membrane peeling.

US 2018/000339 A1 specifies the ascertainment of the model of a patient's eye in an ophthalmological operation on account of data captured intraoperatively, in order to display pieces of information regarding the model in the ophthalmological operation to a surgeon.

In the context of eye surgery visualization systems, it is difficult to spatially correctly display pieces of auxiliary information for a surgeon in a manner over-laid on the observation image of an object region because the coordinates of transparent regions of a patient's eye, for example of regions of the cornea, cannot readily be determined in a coordinate system that is fixed with respect to an eye surgery visualization system.

It is an object of the invention to provide an eye surgery operating system and to specify a computer program and also a method, which increases the precision of the surgical procedures on a patient's eye.

This object is achieved by an eye surgery visualization system for the three-dimensional visualization of an operating site on a patient's eye having the features of claim 1 and by a method having the features of claim 13, and also by a computer program having the features of claim 16. Advantageous embodiments and developments of the invention are set forth in the dependent claims.

A model of the patient's eye is understood by the invention to be a construct that describes only the properties of an original, especially a patient's eye in this case, considered important in order to arrive at an abstracted representation of the original that is manageable or mathematically calculable or suitable for experimental investigations as a result of this simplification.

For example, a model of the patient's eye can be a point cloud that describes the patient's eye. The model of the patient's eye can also describe the surface shape of a cornea of the patient's eye. In particular, a model of the patient's eye can be a CAD model and/or a height profile of a segment of the patient's eye and/or a distance profile of the patient's eye and/or a depth profile of the patient's eye and/or a three-dimensional surface representation of a segment of the patient's eye and/or a two-dimensional surface representation of a segment of the patient's eye.

The invention is based on the idea that the use of three-dimensional biometry data related to a patient's eye, which are registered to a preferably stereoscopic view of the patient's eye, allows for the display of display information with a spatially extended structure, which is overlaid on a displayed stereoscopic image of the patient's eye, in an eye surgery visualization apparatus.

In ophthalmic surgery, procedures are performed inter alia on the anterior and the posterior chamber of a patient's eye. Within the scope of the anterior chamber, the treatment of cataracts is by far the most common operation, with approximately 8 million procedures being carried out each year worldwide. The implanted intraocular lens (IOL) is selected on the basis of biometric data, which are obtained using systems such as e.g. the IOL Master 700 by Carl Zeiss. Intraoperatively, surgical microscopes are used to perform the procedure. In recent years, analog surgical microscopes are increasingly being replaced by hybrid systems.

To increase the precision of surgical procedures on a patient's eye, it is desirable to overlay pieces of information in the form of biometric data, which can be obtained by systems such as e.g. the IOL Master 700 by Carl Zeiss, for instance the size of the capsulorhexis or the axis position of a toric intraocular lens (IOL), in 3-D on a stereoscopic image of the operating site on a patient's eye using simple, cost-effective technologies.

An eye surgery visualization system according to the invention contains a device for the stereoscopic visualization of an object region with an operating site on a patient's eye, the device for the stereoscopic visualization of an object region comprising an image capture device for capturing object region image data containing image data related to at least one patient's eye registration structure. In the eye surgery visualization system there is a computer unit with a computer program, and a biometry data interface for the provision of patient's eye biometry data, which define a patient's eye model having a model registration structure corresponding to the patient's eye registration structure and a model coordinate system. The computer program has a program routine for registering the patient's eye model to the patient's eye on the basis of the patient's eye registration structure and the model registration structure, in which object region image data related to an image of the patient's eye captured by means of the image capture device are linked to optical imaging parameters of the image capture device in order thus to provide in the form of data a patient's eye coordinate system which is fixed in relation to the patient's eye and referenced to the model coordinate system.

This creates an eye surgery visualization system which, by use of image processing and in particular with the aid of technologies that are simple and cost-effective as a matter of principle, is capable of displaying to a surgeon pieces of information such as the size of the capsulorhexis or the axis position of a toric IOL in 3-D at the correct position, in a manner overlaid on an image of a patient's eye.

An operating site on a patient's eye is understood in the present case to mean an operating site which is located in the patient's eye, in the vicinity of the patient's eye or partially within and outside of the patient's eye.

The invention understands object region image data to be data containing information about the position and color of pixels of an image of the object region which is an image representation of the object region. Object region image data can also be data based on processed information with regards to pixels of an image of the object region.

The invention understands image data related to at least one patient's eye registration structure to be image data about the patient's eye registration structure, i.e., image data from an image which is a complete or partial image representation of the patient's eye registration structure.

For example, patient's eye biometry data may contain lengths of structures in the patient's eye determined from OCT data, and may also comprise an eye position image during an OCT image data recording.

The invention understands OCT data to be data provided by an OCT system designed for the capture of structures in body tissue by means of OCT scanning radiation, with the data containing information regarding the spatial position of scattering centers for OCT scanning light, e.g., in body tissue.

It is advantageous if the eye surgery visualization system contains a stereoscopic display device which serves for the display of image information during a stereoscopic visual impression, the computer program comprising a program routine for the calculation of a piece of stereoscopic display information in the patient's eye coordinate system and serving for the provision of display information data which can be displayed on the display device as a spatially extended structure overlaid on the visualized object region.

In the present case, stereoscopic display information is understood to mean display information imparting a spatial impression of depth as it contains image information for the left and the right eye of an observer, with the image information corresponding to a stereoscopic visual impression.

This allows for an eye surgery visualization system to display to a surgeon auxiliary information corresponding to the natural visual impression, and this makes precise spatial movement or the guidance of surgical instruments easier for the surgeon.

The spatially extended structure can be, e.g., a structure from the group of rhexis circle at the location of the capsular bag, IOL axis position, corneal incision.

The invention includes the idea that the patient's eye model describes at least one physiological parameter of the patient's eye from the group of curvature of the cornea, thickness of the cornea, anterior chamber depth, extent of the lens, diameter of the sclera, extent of the iris, extent of the pupil, thickness of the lens, posterior chamber depth, extent of the retina.

The patient's eye registration structure can be, e.g., at least one structure from the group of structure of the iris, structure of the retina, structure of at least one vessel in the sclera, geometry of the white-to-white of the patient's eye.

The device for the stereoscopic visualization of an object region with an operating site on a patient's eye preferably contains an adjustable imaging optical unit with an autofocus system for automatically focusing on a structure in the patient's eye, the computer program having a program routine for evaluating a sharpness of images which are based on object region image data captured by means of the device for capturing object region image data, the program routine being designed to provide control data for the adjustment of a z-focusing of the imaging optical unit.

It is advantageous if the device for the stereoscopic visualization of an object region with an operating site on a patient's eye has an autofocus system for automatically focusing on a point in the patient's eye corresponding to a structure of the patient's eye model or corresponding to a point of interest in the patient's eye model, with the computer program containing a program routine setting a z-focusing and/or an xy-scaling and/or an xy-positioning of the imaging optical unit for sharp imaging of a point or region of the patient's eye corresponding to a point of interest or a model region in the patient's eye model by virtue of, for the imaging optical unit, settings data from coordinates of the point of interest or of the region in the model coordinate system being linked to optical imaging parameters of the device for the stereoscopic visualization of an object region.

The autofocus system may comprise a focusing state interface for the provision of a z-focusing and/or an xy-scaling of the imaging optical unit, and/or an xy-positioning of the device for the capture of stereoscopic image data in a plane perpendicular to an optical axis of a main objective of the imaging optical unit.

It is advantageous here if the computer program obtains at least one piece of information of the group of z-focusing, xy-scaling of the imaging optical unit, xy-positioning from the focusing state interface and obtains patient's eye biometry data from the biometry data interface and takes these into account for the calculation of the display information.

For example, the biometry data interface could be designed to provide patient's eye biometry data captured intraoperatively. The patient's eye biometry data could contain, e.g., OCT data related to the patient's eye and lengths determined from the OCT data related to the patient's eye. In particular, the patient's eye biometry data may comprise an eye position image that underlies the OCT data related to the patient's eye.

It is advantageous if the image capture device is designed for the capture of stereoscopic image data and to this end contains a first image sensor and a second image sensor. The stereoscopic display device may be designed as a 3-D monitor or as a head-mounted device (HMD). It may also be integrated in a binocular tube and in this case comprise a respective display for overlaying image data into a first and into a second stereoscopic partial observation beam path.

In the method according to the invention, object region image data showing at least one registration structure of the patient's eye are captured by means of a device for the capture of object region image data, and patient's eye biometry data defining a patient's eye model with a model coordinate system are provided. In this case, the patient's eye model is registered to the patient's eye by virtue of object region image data related to an image of the patient's eye captured by means of the device being linked to optical imaging parameters of the device and to data of the patient's eye model in order thereby to provide in the form of data a patient's eye coordinate system which is fixed in relation to the patient's eye and referenced to the model coordinate system.

The patient's eye biometry data provided can be ascertained preoperatively or intraoperatively. It should be observed that a point of interest in the biometry data, which define a model of a patient's eye, can be selected and/or determined by a person or using a computer program operating with artificial intelligence. In particular, it should be observed that the provided patient's eye biometry data are intraoperatively captured OCT data related to the patient's eye.

For example, display information data for the display of a spatially extended structure overlaid on the visualized operating site can be provided in the patient's eye coordinate system. It is also possible to provide imaging optical unit settings data for the sharp imaging of a point of interest or region in the patient's eye coordinate system.

Below, the invention will be explained in more detail on the basis of exemplary embodiments depicted schematically in the drawing,

in which:

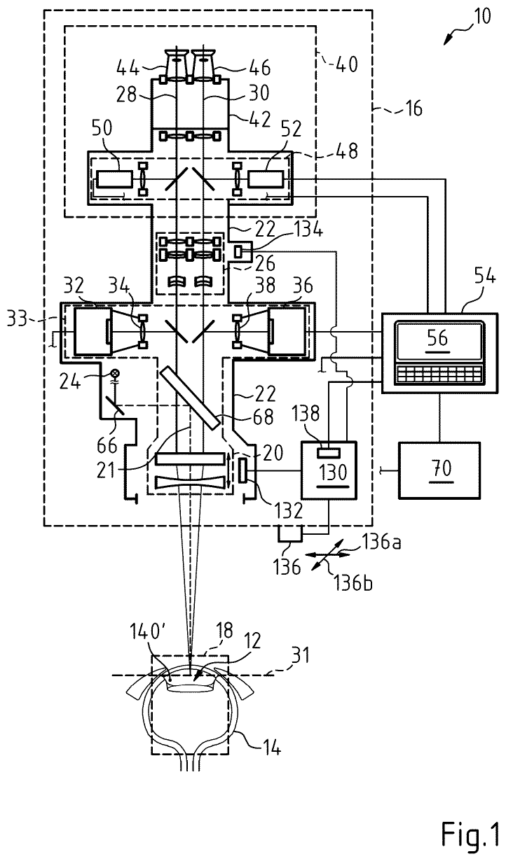

FIG. 1 shows an eye surgery visualization system for the three-dimensional visualization of an operating site on a patient's eye;

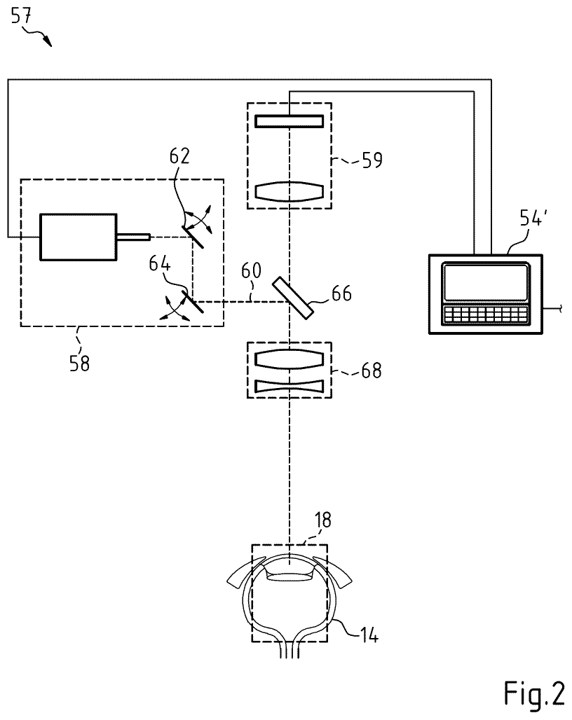

FIG. 2 shows a system for the capture of patient's eye biometry data, which define a model of the patient's eye;

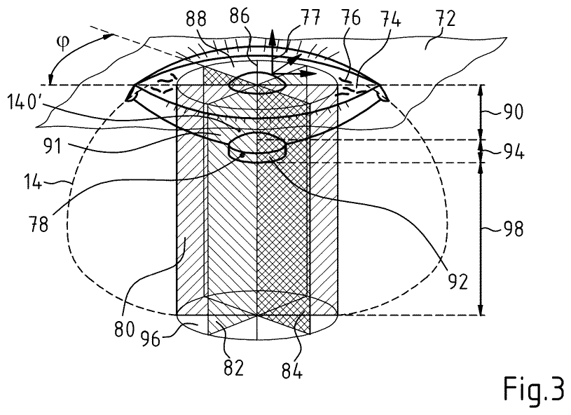

FIG. 3 shows a partial view of the patient's eye with regions in which the system captures patient's eye biometry data;

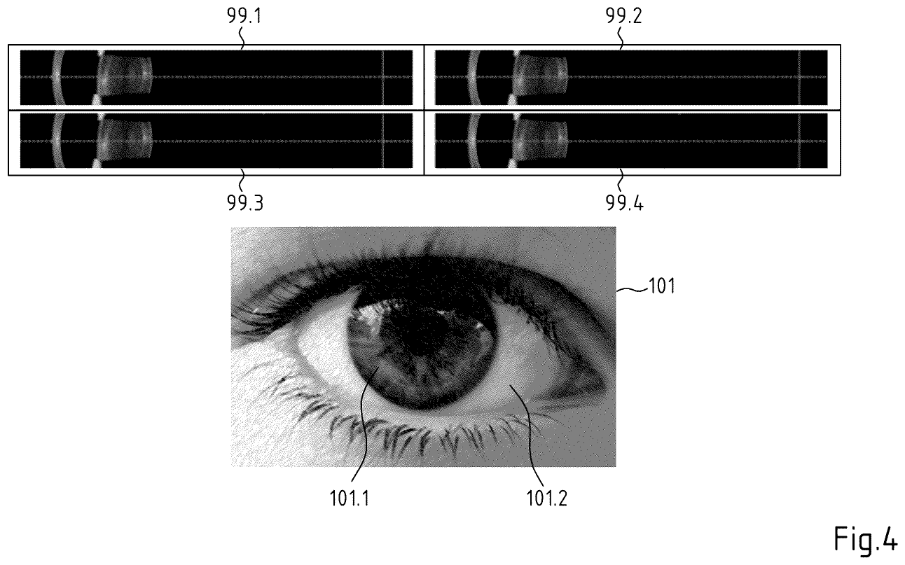

FIG. 4 shows patient's eye biometry data which are provided by the system for the capture of patient's eye biometry data;

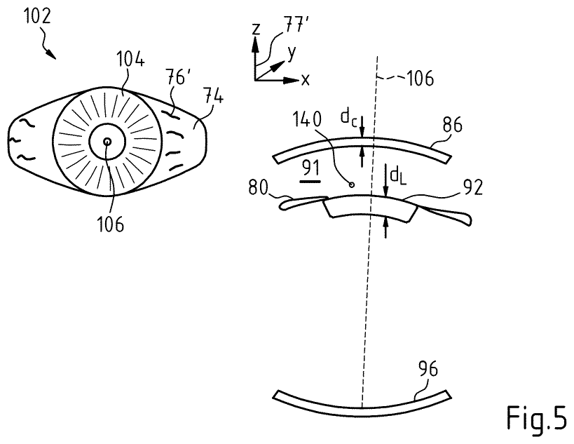

FIG. 5 shows a model of the patient's eye which is defined by the captured patient's eye biometry data;

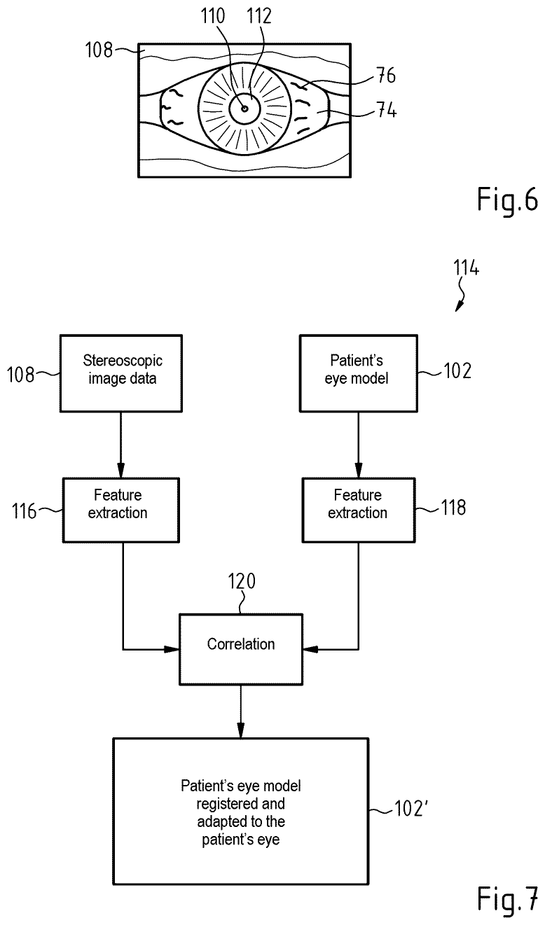

FIG. 6 shows an image of the patient's eye, captured in the eye surgery visualization system;

FIG. 7 shows a flowchart with program routines of a computer program for calculating a piece of stereoscopic display information for display in the eye surgery visualization system, the computer program being loaded into a program memory of a computer unit in the eye surgery visualization system;

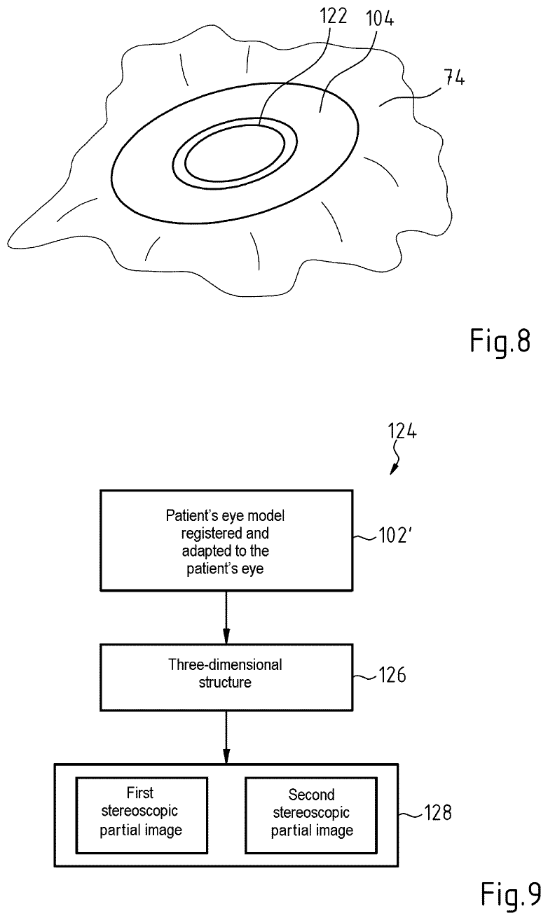

FIG. 8 shows a piece of stereoscopic display information provided in the eye surgery visualization system;

FIG. 9 shows a flowchart with a program routine for calculating stereoscopic display information in the eye surgery visualization system; and

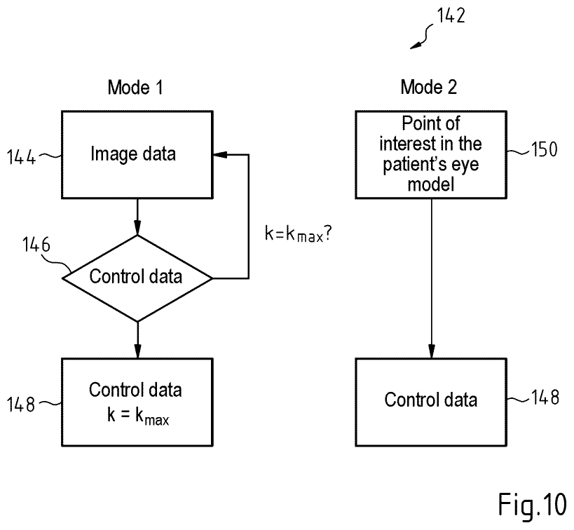

FIG. 10 shows a flowchart with program routines of a computer program for setting a z-focusing, an xy-scaling and an xy-position of the imaging optical unit in the eye surgery visualization system, in order to enable sharp imaging of a region in the patient's eye.

The eye surgery visualization system 10 shown in FIG. 1 serves for the three-dimensional visualization of an object region having an operating site 12 on a patient's eye 14. To this end, the eye surgery visualization system 10 has a device 16 for the stereoscopic visualization of the object region. The device 16 for the stereoscopic visualization of the object region comprises an imaging optical unit having a main objective system 20, the latter having an optical axis 21 and being accommodated in a main body 22. In the device 16 for the stereoscopic visualization of the object region there is an illumination device 24, which allows the operating site 12 to be illuminated by an illumination beam path which passes through the main objective system 20.

The imaging optical unit in the device 16 for the stereoscopic visualization of the object region comprises an afocal magnification system 26, guided through which are a first stereoscopic partial observation beam path 28 and a second stereoscopic partial observation beam path 30.

The main objective system 20 and the magnification system 26 are adjustable by motor and allow a focal plane 31 and a magnification to be set. The main objective system 20 is traversed by the first stereoscopic partial observation beam path 28 and the second stereoscopic partial observation beam path 30.

In the device 16 for the stereoscopic visualization of the object region there is an image capture device 33. The image capture device 33 is designed to capture stereoscopic object region image data which contain image data related to a patient's eye registration structure. To this end, the image capture device 33 comprises a first image sensor 32 with an objective lens system 34, the first image sensor serving for the capture of data with image information from the first stereoscopic partial observation beam path 22, and a second image sensor 36 with an objective lens system 38, in order to capture image information from the second stereoscopic partial observation beam path 30.

The device 16 for the stereoscopic visualization of the object region has an apparatus 40 for the provision of stereoscopic images of the operating site 12. The apparatus 40 contains a binocular tube 42 which is connected to an interface of the main body 22 and has a first binocular eyepiece 44 and a second binocular eyepiece 46. The apparatus 40 comprises a display device 48 for image data overlaid on the stereoscopic partial observation beam paths 28, 30. To this end, the display device 48 contains a display 50 for overlaying image data on the first stereoscopic partial observation beam path 28 and a display 52 which serves to overlay image data on the second stereoscopic partial observation beam path 30.

The eye surgery visualization system 10 has a computer unit 54, which is connected to the image capture device 33 for the capture of stereoscopic object region image data. The computer unit 54 contains a computer program with an image calculation stage, which converts data with image information from the first image sensor 32 and from the second image sensor 36 into spatial image data. The object of the computer unit 54 in the eye surgery visualization system 10 also includes the control of the display device 48 for image data overlaid on the stereoscopic partial observation beam paths 28, 30. The computer unit 54 has a program memory and is connected to an electronic visual display, which serves for displaying a user interface 56.

The eye surgery visualization system 10 contains a biometry data interface 70. The biometry data interface 70 serves for the provision of patient's eye biometry data, which define a patient's eye model having a model registration structure corresponding to the patient's eye registration structure and a model coordinate system.

FIG. 2 shows a system 57 for the capture of such patient's eye biometry data. The system 57 has an OCT apparatus 58 and a camera 59. The OCT apparatus 58 is designed to scan an object region volume on the patient's eye 14 by way of an A-, B-and C-scan. It should be observed that the OCT apparatus can be designed as a so-called SS-OCT apparatus in particular, the latter allowing the object region to be scanned with quasi-low coherent light. To scan the object region volume, an OCT scanning beam 60 which has low-coherent light and can be moved over the object region volume is generated by means of the OCT apparatus 58. The OCT scanning beam 60 serves for the capture of OCT data with spatial image information in the form of image data for slice recordings of the object region volume, as described, e.g., in chapter 3, pages 45 to 82 in A. Ehnes, “Entwicklung eines Schichtsegmentierungsalgorithmus zur automatischen Analyse von individuellen Netzhautschichten in optischen Kohärenztomographie-B Scans”, Dissertation, University of Giessen (2013).

The OCT apparatus 58 has adjustable scanning mirrors 62, 64 serving to move the OCT scanning beam 60. In the eye surgery visualization apparatus 10, the OCT scanning beam 60 is guided into the object region volume on the patient's eye 14 via a beam splitter 66 and a main objective system 68. The light of the OCT scanning beam 60 scattered in the object region volume returns at least in part to the OCT apparatus 58 via the same light path. Then, the light path of the scanning light is compared in the OCT apparatus 58 with a reference path. Using this, patient's eye biometry data in the form of an accurate position of scattering centers in the object region volume can be captured; in particular, said patient's eye biometry data describe the position of optically effective surfaces with an accuracy corresponding to the coherence length lc of the low-coherent light in the OCT scanning beam 60.

The camera 59 is designed to capture an image of the patient's eye which contains the pupil, the iris, and at least some of the sclera with vessels formed therein, which are resolved by the camera and serve as patient's eye biometry data.

The system 57 contains a computer unit 54′, which serves for controlling the OCT scanning beam 60 provided by means of the OCT apparatus 58. The computer unit 54′ allows for setting of the spatial position and orientation of the object region volume in the portion 18 of the patient's eye 14 scanned by the OCT scanning beam 60. The system 57 can supply the captured patient's eye biometry data to the biometry data interface 70 in the eye surgery visualization apparatus 10.

It should be observed that, as a matter of principle, an ophthalmological examination system in the form of the IOL Master 700 by Carl Zeiss can also be used as an alternative to the above-described system for the capture of patient's eye biometry data, which define a patient's eye model, and for the supply of the patient's eye biometry data to the biometry data interface 70 in the eye surgery visualization apparatus 10. In addition to an image of a patient's eye with the pupil, the iris and at least some of the sclera with vessels resolved therein, this system also provides, as patient's eye biometry data, the curvature of the cornea, captured by keratometry, and the position of scattering centers in 6 different OCT scanning planes, which are located on a common optical axis passing through the cornea and the pupil of the patient's eye and azimuthally offset from one another in relation to this axis. It should also be observed that the above-described system 57 for the capture of such patient's eye biometry data may also be integrated in an eye surgery visualization system 10 as shown in FIG. 1.

FIG. 3 shows a partial view of the patient's eye 14 with a patient's eye coordinate system 77 that is fixed in relation to the patient's eye 14 and with regions in which the system 57 captures patient's eye biometry data. In this case, first patient's eye biometry data captured by means of the system 57 are pixels in an image plane 72 with the sclera 74 and vessels formed therein as a patient's eye registration structure 76. Second patient's eye biometry data captured by means of the system 57 are in this case scattering centers 78 for the OCT scanning beam in the OCT scanning planes 80, 82, 84 and 86 which are offset from one another by the azimuth angle φ=45°. These scattering centers 78 specify the position of the cornea 88, its thickness, the depth 90 of the anterior chamber 91, the position of the lens 92, the thickness 94 of the latter and the position of the retina 96, and also the depth 98 of the posterior chamber 100 in the patient's eye 14.

FIG. 4 shows patient's eye biometry data which are provided by the system 57 for the capture of patient's eye biometry data. These patient's eye biometry data comprise OCT scanning data 99.1, 99.2, 99.3, and 99.4 in the form of the position of scattering centers for the OCT scanning light in the OCT scanning planes 80, 82, 84, and 86 of FIG. 3, and an image 101 of the patient's eye captured by means of the camera 59, the image showing the iris 101.1 and the white-to-white 101.2 as a portion of the sclera 74.

FIG. 5 shows a patient's eye model 102 which defines the patient's eye biometry data captured by means of the system 57: In addition to the position and structure of vessels in the plane of the sclera as a model registration structure 76′, parameters of this model include the spatial position and extent of the cornea 86, the thickness dc of the cornea 86, the spatial position and extent of the anterior chamber 91, the spatial position and extent of the lens 92, the lens thickness d_{L}, the spatial position and extent of the iris 106, the structure of vessels 76 in the sclera 74, and the spatial position and extent of the retina 96. It should be observed that the structure and the position of the iris 104 in the patient's eye 14 can also be a parameter of the model 102 of the patient's eye 14.

The patient's eye model 102 defines at least a spatial extent of a portion of the patient's eye 14 and a model coordinate system 77′. To this end, the model 102 has the property that it models the course of an optical axis 106 for the lens 92 in the patient's eye 14 and the position and extent of at least one structure of a patient's eye registration structure 76 in the form of a vessel of the sclera 74, and/or at least one structure of the iris 104 in relation to the optical axis 106. On account of this definition, the patient's eye model 102 can be uniquely referenced on the basis of the position and spatial extent of the at least one vessel of the sclera 74 and/or the at least one structure of the iris 104 in relation to the patient's eye 14 on the basis of an image of the patient's eye 14 captured in the above-described eye surgery visualization system 10 in the case of a known magnification and known position of the focal plane as optical imaging parameters, the image containing the at least one vessel of the sclera 74 or the at least one structure of the iris 104.

FIG. 6 shows an image 108 of the patient's eye 14 captured on the first image sensor 32 in the eye surgery visualization system 10. The computer unit 54 in the eye surgery visualization system 10 contains a computer program with a program routine for registering the patient's eye model 102 of the patient's eye 14, which was defined by the patient's eye biometry data provided at the biometry data interface 70, to the patient's eye 14 in the eye surgery visualization system 10.

In this case, the computer program determines by means of image processing the center 110 of the pupil 112 of the patient's eye and, in the image 112, the position and spatial extent of the at least one vessel 76 of the sclera 74 contained in the model 102 of the patient's eye and/or the at least one structure of the iris 104 contained in the model 102 of the patient's eye.

In this case, the computer program uses vessels in the sclera as non-transparent structures in the patient's eye. In principle, it is also possible to reference a three-dimensional model of the patient's eye to the patient's eye on the basis of the structures in the iris of the patient's eye.

Using the optical imaging parameters underlying the image 108 of the patient's eye 14, the computer program then registers the patient's eye model 102 to the patient's eye 14 on the basis of the image 108 by virtue of making the patient's eye registration structure 76 consistent with the model registration structure 76′. In this respect, it is also possible to take account of an ascertained center 110 of the pupil 112 and an ascertained position and structure in the iris of the patient's eye 14.

FIG. 7 shows a flowchart 114 of the program routine for registering the patient's eye model 102 of the patient's eye 14, which was defined by the patient's eye biometry data provided at the biometry data interface 70, to the patient's eye 14 in the eye surgery visualization system 10.

To this end, the program routine contains a routine 116 for extracting characteristic features of the image data in the image 108 as a patient's eye registration structure 76 in the form of the structure of a vessel in the sclera 74 and/or a structure of the iris 104, and a routine 118 for extracting at least one corresponding model registration structure 76′ in the patient's eye model 102.

The program routine has a correlation routine 120, which correlates mutually corresponding characteristic features of the image data of the image 108 and the characteristic features of the patient's eye model 102 taking account of the optical imaging parameters underlying the image 108 of the patient's eye 14, and hence links the object region image data captured by means of the device 33 for the capture of stereoscopic object region image data to data of the patient's eye model 102 and to the optical imaging parameters. The program routine then provides a patient's eye model 102′ of the patient's eye 14 which is registered and adapted to the patient's eye 14 in the eye surgery visualization system 10, wherein the patient's eye coordinate system 77 and the model coordinate system 77′ are referenced to one another.

The computer program allows the calculation of a piece of stereoscopic display information from the model 102 of the patient's eye 14 registered to the patient's eye 14, for the display device 48 in the device 16 for the stereoscopic visualization of the object region in the eye surgery visualization system 10. To this end, the computer program comprises a program routine for calculating the piece of stereoscopic display information.

FIG. 8 shows the calculated piece of stereoscopic display information in the form of a spatially extended structure 122, which is in the form of a rhexis circle at the location of the capsular bag and overlaid on the stereoscopic image data with the iris 104 and with the sclera 74, it being possible to display this structure by means of the display device 48 for the provision of stereoscopic images. It should be observed that, e.g., an IOL axis position or a corneal incision can also be displayed as a spatially extended structure 120 that is overlaid on the stereoscopic image data.

FIG. 9 shows a flowchart 124 of the program routine for calculating stereoscopic display information in the eye surgery visualization system.

From the patient's eye model 102′ registered to the patient's eye 14, the routine in a step 126 calculates a three-dimensional structure, for example in the form of the rhexis circle, in the patient's eye coordinate system 77 which is referenced to the model coordinate system 77′, this three-dimensional structure being a piece of auxiliary information for a surgeon who performs a surgical procedure on the patient's eye 14 using the eye surgery visualization apparatus 10.

Then, in a step 128, stereoscopic image data for the display of the three-dimensional structure by the display device 48 are then calculated for this three-dimensional structure as a stereoscopic image in the first and the second partial observation beam path 28, 30 by means of the displays 50, 52. To this end, a first stereoscopic partial image is specified for the display 50 and a second stereoscopic partial image is specified for the display 52 in the step 126; the second stereoscopic partial image has such a disparity with respect to the first stereoscopic partial image that a stereoscopic visual impression of the three-dimensional structure overlaid on the image of the operating site 12, which corresponds to the stereoscopic visual impression of the operating site 12, is effected for an observer who observes the operating site 12 through the first and second binocular eyepiece 44, 46 of the binocular tube 42.

The eye surgery visualization system 10 shown in FIG. 1 contains an autofocus system 130 which is connected to the computing unit 54 and which is linked to a servomotor 132 for adjusting the main objective system 20 and to a servomotor 134 for adjusting the magnification system 26. The eye surgery visualization system 10 contains an xy-adjustment unit 136 driven by a motor, the latter serving to displace the device 16 for the capture of stereoscopic image data in a plane perpendicular to the optical axis 21 of the main objective system 20, in accordance with arrows 136a, 136b. The autofocus system 130 has a focusing state interface 138 for providing a z-focusing and an xy-scaling and an xy-positioning of the imaging optical unit in the device 16 for the capture of stereoscopic image data.

The autofocus system 130 can be operated in a first mode which enables automatic focusing on a structure in the patient's eye 14 in the form of the iris or the sclera. To this end, an image of the patient's eye 14 captured by means of the first or second image sensor 32, 36 is evaluated in respect of sharpness, and the servomotor 100 is adjusted by means of the autofocus system 98 for maximum image sharpness.

In a second mode, the autofocus system 130 enables automatic focusing on a point 140′ in the patient's eye 14, as shown in FIG. 3, said point corresponding to a structure in the model 102 of the patient's eye 14 or to a point of interest 140 of the patient's eye model 102 shown in FIG. 9. For automatic focusing on a location 140′ in the patient's eye 14 corresponding to a structure in the model 102 of the patient's eye 14 or to a point of interest 140 in the model 102 of the patient's eye, the computer program of the computer unit 54 contains a program routine which serves for the setting of a z-focusing, an xy-scaling, and an xy-positioning of the imaging optical unit in the device 16 for the stereoscopic visualization of an object region, in order to enable sharp imaging of the point 140′ in the patient's eye 14.

FIG. 10 is a flowchart 142 of this program routine, which can be operated in two different modes.

In the first mode, the image data captured by means of the first image sensor 32 are evaluated in respect of sharpness in a step 144, this evaluation being based on the determination of the contrast k in a specifiable image region of the image underlying the image data. In a step 146, control data for the servomotor 132 for z-focusing, the servomotor 134 for the xy-scaling, and the servomotor for the xy-positioning of the imaging optical unit are varied by means of the xy-adjustment unit 136 in the eye surgery visualization system 10 such that the contrast k determined in step 144 is at a maximum. The control data for the maximum contrast kₘₐₓ then correspond to the sought-after control data 148 for a setting for the z-focusing, the xy-scaling, and the xy-positioning of the imaging optical unit in the eye surgery visualization system 10.

In the second mode, control data for the z-focusing, the xy-scaling, and the xy-positioning of the imaging optical unit in the eye surgery visualization system 10 are calculated in a step 150 in the model coordinate system 77′ for a point of interest 140, shown in FIG. 10, in the patient's eye model 102′ registered and adapted to the patient's eye 14, the calculation being implemented by virtue of settings data from the coordinates of the point of interest 140 or the region in the model coordinate system 77′ being linked for the imaging optical unit with optical imaging parameters of the device 16 for the stereoscopic visualization of an object region.

In the patient's eye 14 shown in FIG. 3, these control data cause the location 140′ corresponding to the point of interest 140 in FIG. 10 to be located on the optical axis of the main objective system 20 of the eye surgery visualization system 10 and in the focal plane of the main objective system 20, wherein the xy-scaling is set to a constant value by tracking the magnification system 26.

In conclusion, the following preferred features should, in particular, be retained: An eye surgery visualization system 10 has a device 16 for the stereoscopic visualization of an object region with an operating site 12 on a patient's eye 14, the device 16 for the stereoscopic visualization of an object region comprising an image capture device 33 for capturing object region image data containing image data related to at least one patient's eye registration structure 76. The eye surgery visualization system 10 comprises a computer unit 54 with a computer program, and contains a biometry data interface 70 for the provision of patient's eye biometry data, which define a patient's eye model 102 having a model registration structure 76′ corresponding to the patient's eye registration structure 76 and a model coordinate system 77′. The computer program has a program routine for registering the patient's eye model 102 to the patient's eye 14 on the basis of the patient's eye registration structure 76 and the model registration structure 76′, in which object region image data related to an image of the patient's eye 14 captured by means of the image capture device 33 are linked to optical imaging parameters of the image capture device 33 in order thus to provide in the form of data a patient's eye coordinate system 77 which is fixed in relation to the patient's eye 14 and referenced to the model coordinate system 77′.

### LIST OF REFERENCE SIGNS

10 Eye surgery visualization system
12 Operating site
14 Patient's eye
16 Device for the capture of stereoscopic image data
18 Portion of the patient's eye
20 Main objective system
21 Optical axis
22 Main body
24 Illumination device
26 Magnification system
28 First stereoscopic partial observation beam path
30 Second stereoscopic partial observation beam path
31 Focal plane
32 First image sensor
33 Image capture device
34 Objective lens system
36 Second image sensor
38 Objective lens system
40 Apparatus for the provision of stereoscopic images
42 Binocular tube
44 First binocular eyepiece
46 Second binocular eyepiece
48 Display device
50 Display for overlaying image data on the first stereoscopic beam path
52 Display for overlaying image data on the second stereoscopic beam path
54, 54′ Computer unit
56 User interface
57 System for the capture of patient's eye biometry data
58 OCT apparatus
59 Camera
60 OCT scanning beam
62, 64 Scanning mirror
66 Beam splitter
68 Main objective system
70 Biometry data interface
72 Image plane
74 Sclera
76 Model registration structure
77 Patient's eye coordinate system
77′ Model coordinate system
78 Scattering center for the OCT scanning beam
80, 82, 84, 86 OCT scanning plane
88 Cornea
90 Anterior chamber depth
91 Anterior chamber
92 Lens element
94 Lens thickness
96 Retina
98 Posterior chamber depth
99.1, 99.2, 99.3, 99.4 Patient's eye biometry data
100 Posterior chamber
101 Image of the patient's eye
101.1 Iris
101.2 White-to-white
102 Patient's eye model
102′ Adapted patient's eye model
104 Iris
106 Optical axis
108 Image
110 Center of the pupil
112 Pupil
114 Flowchart
116, 118 Routine
120 Correlation routine
122 Structure
124 Flowchart
126, 128 Step
130 Autofocus system
132, 134 Servomotor
136 xy-adjustment unit
136a, 136b Arrow
138 Focusing state interface
140 Point of interest
140′ Point corresponding to the point of interest
142 Flowchart
144, 146, 150 Step
148 Control data

1-16. (canceled)

17. An eye surgery visualization system,
having a device for the stereoscopic visualization of an object region with an operating site on a patient's eye, the device for the stereoscopic visualization of an object region comprising an image capture device for capturing object region image data containing image data related to at least one patient's eye registration structure,
having a computer unit, and
having a biometry data interface for the provision of patient's eye biometry data, which define a patient's eye model having a model registration structure corresponding to the patient's eye registration structure and a model coordinate system,
having a stereoscopic display device which serves for the display of image information with a stereoscopic visual impression,
wherein
the computer unit contains a computer program with a program routine for registering the patient's eye model to the patient's eye on the basis of the patient's eye registration structure and the model registration structure, in which the object region image data captured by means of the image capture device are linked to optical imaging parameters of the image capture device in order thus to provide in the form of data a patient's eye coordinate system which is fixed in relation to the patient's eye and referenced to the model coordinate system,
said computer program comprising a program routine for the calculation of stereoscopic display information in the patient's eye coordinate system, said program routine serving for the provision of display information data which can be displayed on the display device as a spatially extended structure overlaid on an object region visualized by said device for the stereoscopic visualization of an object region,
wherein said device for the stereoscopic visualization of an object region contains an adjustable imaging optical unit with an autofocus system,
wherein the autofocus system comprises a focusing state interface for the provision of a z-focusing and/or an xy-scaling of the imaging optical unit and an xy-positioning of the device for the capture of stereoscopic image data in a plane perpendicular to an optical axis of a main objective of the imaging optical unit,
and wherein the computer program obtains the z-focusing and/or the xy-scaling of the imaging optical unit and/or the xy-positioning from the focusing state interface and obtains patient's eye biometry data from the biometry data interface and takes these into account for the calculation of the stereoscopic display information and the provision of display information data which can be displayed on the display device as a spatially extended structure overlaid on the object region.

18. The eye surgery visualization system as claimed in claim 17, wherein the spatially extended structure is a structure from the group of rhexis circle at the location of the capsular bag, IOL axis position, corneal incision.

19. The eye surgery visualization system as claimed in claim 17, wherein the patient's eye model describes at least one physiological parameter of the patient's eye from the group of curvature of the cornea, thickness of the cornea, anterior chamber depth, extent of the lens, diameter of the sclera, extent of the iris, extent of the pupil, thickness of the lens, posterior chamber depth, extent of the retina and/or wherein the patient's eye registration structure is at least one structure from the group of structure of the iris, structure of the retina, structure of at least one vessel in the sclera, geometry of the white-to-white of the patient's eye.

20. The eye surgery visualization system as claimed in claim 17, wherein the device for the stereoscopic visualization of an object region contains an adjustable imaging optical unit with an autofocus system for automatically focusing on a structure in the patient's eye, the computer program having a program routine for evaluating a sharpness of images which are based on object region image data captured by means of the image capture device, the program routine being designed to provide control data for the adjustment of a z-focusing of the imaging optical unit.

21. The eye surgery visualization system as claimed in claim 17, wherein the autofocus system is adapted for automatically focusing on a point in the patient's eye corresponding to a structure of the patient's eye model or corresponding to a point of interest in the patient's eye model, with the computer program containing a program routine setting a z-focusing and/or an xy-scaling and/or an xy-positioning of the imaging optical unit for sharp imaging of a point or region of the patient's eye corresponding to a point of interest or a model region in the patient's eye model by virtue of, for the imaging optical unit, settings data from coordinates of the point of interest or of the region in the model coordinate system being linked to optical imaging parameters of the device for the stereoscopic visualization of an object region.

22. The eye surgery visualization system as claimed in claim 17, wherein the biometry data interface is designed for the provision of intraoperatively captured patient's eye biometry data.

23. The eye surgery visualization system as claimed in claim 17, wherein the patient's eye biometry data contain OCT data related to the patient's eye and/or lengths determined from the OCT data related to the patient's eye.

24. The eye surgery visualization system as claimed in claim 23, wherein the patient's eye biometry data comprise an eye position image that underlies the OCT data related to the patient's eye.

25. The eye surgery visualization system as claimed in claim 17, wherein the image capture device is designed for the capture of stereoscopic image data and to this end contains a first image sensor and a second image sensor.

26. A method for operating an eye surgery visualization system having a device for the stereoscopic visualization of an object region with an operating site on a patient's eye, said device including an image capture device and an adjustable imaging optical unit with an autofocus system, wherein the autofocus system comprises a focusing state interface for the provision of a z-focusing and/or an xy-scaling of the imaging optical unit and an xy-positioning of the device in a plane perpendicular to an optical axis of a main objective of the imaging optical unit,
in which
an object region with an operating site on a patient's eye is visualized,
in which object region image data showing at least one registration structure of the patient's eye are captured by means of an image capture device, and in which patient's eye biometry data defining a patient's eye model with a model coordinate system are provided,
wherein
the patient's eye model is registered to the patient's eye on the basis of the patient's eye registration structure and the model registration structure by virtue of linking the object region image data to optical imaging parameters of the image capture device in order thus to provide in the form of data a patient's eye coordinate system which is fixed in relation to the patient's eye and referenced to the model coordinate system, wherein a z-focusing and/or an xy-scaling of the imaging optical unit and an xy-positioning of the device for the capture of stereoscopic image data in a plane perpendicular to an optical axis of a main objective of the imaging optical unit are obtained, and
wherein the patient's eye biometry data and the z-focusing and/or the xy-scaling of the imaging optical unit and the xy-positioning of the device for the capture of stereoscopic image data in a plane perpendicular to an optical axis of a main objective of the imaging optical unit are taken into account for the calculation of the stereoscopic display information and the provision of the display information data for the stereoscopic display of a spatially extended structure which is overlaid on the visualized operating site is provided in the patient's eye coordinate system.

27. The method of clam 26, wherein z-focusing and xy-positioning of the device for the capture of stereoscopic image data in a plane perpendicular to an optical axis of a main objective of the imaging optical unit imaging optical unit settings data are provided for the sharp imaging of a point of interest or region in the patient's eye coordinate system.

28. A non-transitory computer-readable storage medium storing a computer program for carrying out all method steps specified in claim 27 on a computer unit.

29. A non-transitory computer-readable storage medium storing a computer program for carrying out all method steps specified in claim 26 on a computer unit.
